(19) United States
(12) Patent Application Publication
(10) Pub. No.: US 2021/0333539 A1
(43) Pub. Date: Oct. 28, 2021

(54) ENDOSCOPE DISTAL END STRUCTURE AND ENDOSCOPE

(71) Applicant: OLYMPUS CORPORATION, Tokyo (JP)

(72) Inventor: Toru WAKABAYASHI, Tokyo (JP)

(73) Assignee: OLYMPUS CORPORATION, Tokyo (JP)

(21) Appl. No.: 17/371,314

(22) Filed: Jul. 9, 2021

Related U.S. Application Data
(63) Continuation of application No. PCT/JP2019/001063, filed on Jan. 16, 2019.

(51) Int. Cl. G02B 23/24

(52) U.S. Cl. CPC G02B 23/2484; G02B 23/2423

(57) ABSTRACT

An endoscope distal end structure includes: an imaging module including an optical unit, an imaging element, a circuit substrate, as electronic component, a cable, a resin sealing portion configured to seal from an electronic-component mounting region of the circuit substrate to a mounting region of the cable, and a projection portion protruding from as outer periphery of the resin sealing portion; a frame body including a through hole penetrating in an optical axis direction of the optical unit and having a partially opened side surface and configured to hold the imaging module while inserted in the through hole from an insertion port; a first adhesive bonding the projection portion and the opened side surface of the through hole; and a second adhesive filled in a gap between the through hole and the imaging module inserted in the through hole and bonding the frame body and the imaging module.

This application is a continuation of International Application No. PCT/JP2019/001063, filed on Jan. 16, 2019, the entire contents of which are incorporated herein by reference.

### BACKGROUND

The present disclosure relates to an endoscope distal end structure and an endoscope.

In the related art, an endoscope acquires, thorough inserting an elongated flexible insertion portion having an imaging module at its distal end into a subject such as a patient, image data in the subject with the imaging module disposed at the distal end portion and sends the image data to an external information processing device. The imaging module is inserted into a metallic frame body from the standpoint of protecting an imaging element, and an adhesive made of a thermosetting resin or the like is injected inside the frame body to reduce stress applied to the imaging element, alleviate the influence of moisture and the like, and fix the position of the imaging element.

### SUMMARY

According to one aspect of the present disclosure, there is provide an endoscope distal end structure including: an imaging module including an optical unit, an imaging element, a circuit substrate, an electronic component, a cable, a resin sealing portion configured to seal from an electronic-component mounting region of the circuit substrate to a mounting region of the cable, and a projection portion protruding from an outer periphery of the resin sealing portion; a frame body including a through hole, the through hole penetrating in an optical axis direction of the optical unit and having a partially opened side surface, and the through hole being configured to hold the imaging module in a state of being inserted in the through hole from an insertion port disposed at a proximal end portion of the through hole; a first adhesive bonding the projection portion and the opened side surface of the through hole; and a second adhesive filled in a gap between the through hole of the frame body and the imaging module inserted in the through hole and bonding the frame body and the imaging module.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 illustrates a schematic view of the entire configuration of an endoscope system according to an embodiment;

FIG. 2 illustrates a perspective view of an endoscope distal end structure used in the endoscope system in FIG. 1;

FIG. 3 illustrates a top view of an imaging module used in the endoscope distal end structure in FIG. 2;

FIG. 4A illustrates a top view and FIG. 4B illustrates a cross sectional view taken along the line A-A of the endoscope distal end structure in FIG. 2;

FIG. 5A illustrates a top view and FIG. 5B illustrates a cross sectional view taken along the line A-A of as endoscope distal end structure according to a modification example 1 of the embodiment;

FIG. 6A illustrates a top view and FIG. 6B illustrates a cross sectional view taken along the line A-A of as imaging module according to a modification example 2 of the embodiment.

FIG. 7A illustrates a top view and FIG. 7B illustrates a cross sectional view taken along the line A-A of an imaging module according to a modification example 3 of the embodiment.

FIG. 8A illustrates a top view and FIG. 8B illustrates a cross sectional view taken along the line A-A of an imaging module according to a modification example 4 of the embodiment.

FIG. 9A illustrates a top view and FIG. 9B illustrates a cross sectional view taken along the line A-A of an endoscope distal end structure according to a modification example 5 of the embodiment;

### DETAILED DESCRIPTION

An endoscope system including an endoscope distal end structure will be described below as a mode for carrying out the present disclosure (hereinafter referred to as an “embodiment”). Note that the present disclosure is not limited by the following embodiment. Further, in each drawing referred to in the following description, a shape, a size, and a positional relation are only schematically illustrated to an extent that allows contents to be understood. Therefore, the present disclosure is not limited only to the shape, the size, and the positional relation represented in each drawing. Moreover, there are parts where dimensions and ratios are different between the drawings.

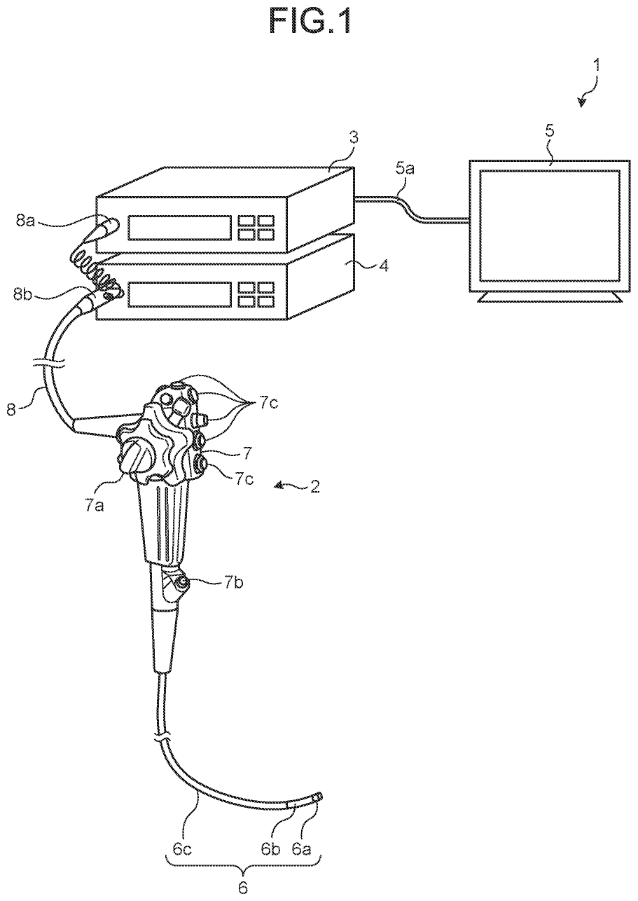

FIG. 1 illustrates a schematic view of the entire configuration of an endoscope system 1 according to a first embodiment. As illustrated in FIG. 1, an endoscope system 1 according to a present embodiment includes an endoscope 2 that is introduced in a subject and generates an image signal in the subject by capturing an image of the inside of the body of the subject, an information processing device 3 that applies prescribed image processing to the image signal captured by the endoscope 2 and controls each unit of the endoscope system 1, an light source device 4 that generates illumination light of the endoscope 2, and a display device 5 that displays an image of the image signal which has been subjected to the image processing by the information processing device 3.

The endoscope 2 includes an insertion portion 6 that is inserted in the subject, an operating unit 7 that is disposed on the proximal end portion side of the insertion portion 6 and held by an operator, and a flexible universal cord 8 that is extended from the operating unit 7.

The insertion portion 6 may be obtained by using a light guide constituted by an illumination fiber, an electric cable, an optical fiber, or the like. The insertion portion 6 includes a distal end portion 6a incorporating an image device described below, a freely bendable bending portion 6b that is configured by multiple bending pieces, a flexible tube portion 6c having flexibility disposed on the proximal end portion side of the bending portion 6b. The distal end portion 6a includes an illumination unit that illuminates the inside of the subject through an illumination lens, an observation unit that captures an image of the inside of the subject, an opening portion that communicates with a treatment tool channel, and an air-supply/water supply nozzle (not illustrated).

The operating unit 7 includes a bending knob 7a that bends the bending portion 6b in a vertical direction and a horizontal direction, a treatment tool insertion unit 7b from which a treatment tool such as biological forceps or a laser knife is inserted into a body cavity of the subject, and a plurality of switch units 7c that operate peripheral devices such as the information processing device 3, the light source device 4, an air supply device, a water supply device, and a gas supply device. The treatment tool which is inserted from the treatment tool insertion unit 7b is exposed from an opening at the distal end of the insertion portion 6 through the treatment tool channel disposed inside the insertion portion 6.

The universal code 8 is configured by using a light guide constituted by an illumination fiber, a cable, or the like. The universal code 8 is branched at the proximal end, and one branched end portion functions as a connector 8a and the other proximal end functions as a connector 8b. The connector 8a is detachably connected to a connector of the information processing device 3. The connector 8b is detachably connected to the light source device 4. The universal code 8 allows illumination light emitted from the light source device 4 to be transmitted to the distal end portion 6a via the connector 8b and the light guide constituted by the illumination fiber. Further, the universal code 8 allows the image signal captured by the image device described below to be transmitted to the information processing device 3 via the cable and the connector 8a.

The information processing device 3 applies prescribed image processing to the image signal outputted from the connector 8a and controls the entire endoscope system 1.

The light source device 4 is configured by using a light source that emits light, a condenser lens, and the like. The light source device 4 supplies light emitted from the light source to the endoscope 2 connected via the connector 8b and the light guide constituted by the illumination fiber of the universal code 8 as illumination light illuminating the inside of the subject being an object under the control of the information processing device 3.

The display device 5 is configured by using a display device employing a liquid crystal or an organic EL (electro luminescence), or the like. The display device 5 displays various pieces of information including an image which has been subjected to the prescribed image processing by the information processing device 3 via an image cable 5a. In this configuration, the operator may make an observation at a desired position inside the subject and determine a symptom by manipulating the endoscope 2 while viewing the image (in-vivo image) displayed by the display device 5.

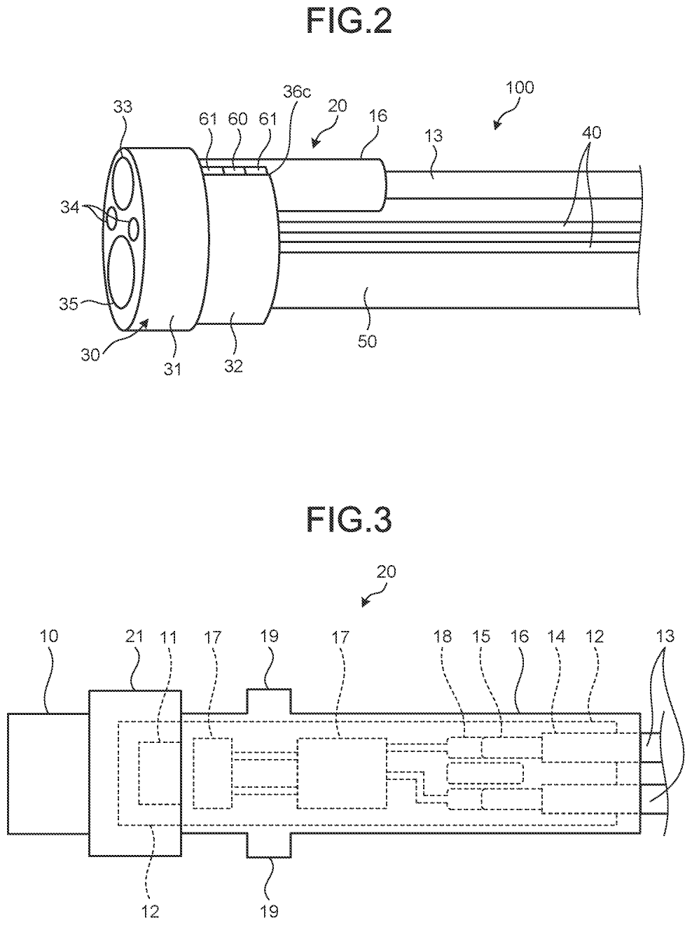

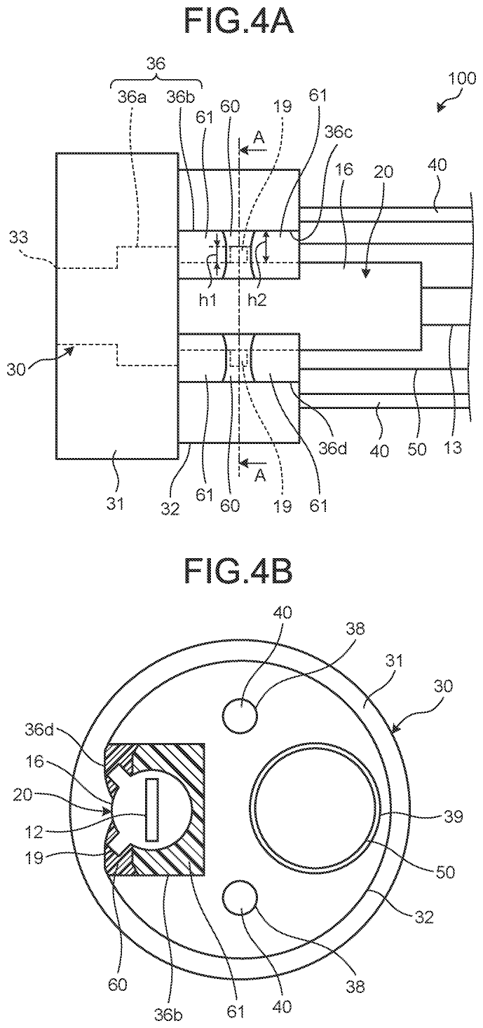

Next, an endoscope distal end structure disposed at the distal end portion 6a of the endoscope 2 will be described in detail. FIG. 2 illustrates a perspective view of an endoscope distal end structure 100 used in the endoscope system 1 in FIG. 1. FIG. 3 illustrates a top view of an imaging module 20 used in the endoscope distal end structure 100 in FIG. 2. FIG. 4A illustrates a top view and FIG. 4B illustrates a cross sectional view taken along the line A-A in FIG. 4A of the endoscope distal end structure 100 according to an embodiment.

An endoscope distal end structure 100 includes an imaging module 20, a frame body 30 that includes a through hole 36, which penetrates in the optical axis direction of an optical unit 10 and has a partially opened side surface, and holds the imaging module 20 in a state of being inserted in the through hole 36 from an insertion port 36c (illustrated in FIG. 4, but not illustrated in FIG. 2) disposed at the proximal end portion of the through hole 36, a first adhesive 60 that bonds projection portions 19 and the opened side surface of the through hole 36, and a second adhesive 61 that is filled in a can between the through hole 36 of the frame body 30 and the imaging module 20 inserted in the through hole 36 and bonds the frame body 30 and the imaging module 20.

The imaging module 20 includes the optical unit 10 that forms an image of an object, an imaging element 11 that photoelectrically converts the image of the object formed by the optical unit 10 to generate an image signal, a circuit substrate 12 mounted with the imaging element 11 and an electronic component 17, cables 13 connected to the circuit substrate 12, a resin sealing portion 16 that seals from an electronic-component mounting region of the circuit substrate 12 to a mounting region of the cables 13, and the projection portions 19 that protrude from the outer periphery of the resin sealing portion 16.

The optical unit 10 includes a plurality of objective lenses not illustrated and a lens holder that holds a cover glass.

The imaging element 11 is configured by CCD, CMOS, or the like, and a light receiving unit of the imaging element 11 is covered by the cover glass and bonded thereto. The imaging element 11 is held by an imaging element frame 21, and the imaging element frame 21 is fitted to the lens holder of the optical unit 10.

A wiring pattern 18 and connection electrodes are formed on the surface of the circuit substrate 12, and the connection electrodes are electrically and mechanically connected to the imaging element 11, the electronic component 17 that drives the imaging element 11, and core wires 15 of the multiple cables 13 that supply a power source to the imaging element 11 or input or output a signal to the imaging element 11. Sheaths 14 at the end portions of the cables 13 are removed to expose the core wires.

The resin sealing portion 16 seals from seals from The electronic-component mounting region of the circuit substrate 12 to the mounting region of the cables 13. The resin sealing portion 16 protects a connection portion between the electronic component 17 and the circuit substrate 12 and a connection portion between the circuit substrate 12 and the cables 13.

Two projection portions 19 are formed on the outer periphery of the resin sealing portion 16 visually recognizable from the partially opened side surface of the through hole 36 at the same position in the optical axis direction of the outer periphery of the resin sealing portion 16 so as to be opposed to the side surface of the through hole 36. The projection portions 19 are made of the same resin as the resin sealing portion 16 and formed by injection molding by using a mold having a shape of the resin sealing portion 16 and the projection portions 19. The projection portions 19 are formed in a rectangular column shape. However, they may be formed in a cylinder shape, a hemispherical shape, a conical shape, or a pyramid shape. A distance h1 of the projection portions 19 is preferably 50% to 70% of a distance h2 of a gap between a through hole 36b and the resin sealing portion 16 from the standpoints of ease of inserting the imaging module 20 into the through hole 36b and reducing the use amount of the first adhesive 60.

The frame body 30 is constituted by a distal end frame portion 31 and a proximal end frame portion 32, and the proximal end frame portion 32 covered by a coated tube is formed in a smaller diameter than the distal end frame portion 31. The frame body 30 includes through holes 36, 38, and 39, and the imaging module 20, a light guide 40, and a channel tube 50 for a treatment tool or the like are inserted into the through holes 36, 38, and 39, respectively, so that the imaging module 20, the channel tube 50 for the treatment tool or the like, and the light Guide 40 are held and fixed by the frame body 30. An observation window 33, an illumination window 34, and a treatment tool port 35 are provided on the distal end surface of the frame body 30.

The through hole 36 is provided so as to penetrate from the distal end frame portion 31 to the proximal end frame portion 32 in the optical axis direction. A through hole 36a inside the distal end frame portion 31 is formed in a cylindrical shape haying a concentric step portion to which the optical unit 10 and the imaging element 11 are fitted. The through hole 36b inside the proximal end frame portion 32 is formed in a rectangular column shape which is larger than the shape of the resin sealing portion 16. In the proximal end frame portion 32, the side surface on the outer periphery side of the through hole 36b is removed, resulting in an opening of the through hole 36b. Providing an opening portion 36d to the proximal end frame portion 32 allows the second adhesive 61 to be easily filled into the through hole 36.

The first adhesive 60 bonds the projection portions 19 and the opened side surface of the through hole 36b. As shown in FIG. 4E, the first adhesive 60 causes the imaging module 20 to be adhesively fixed to the frame body 30 in a state of covering the surface of the projection portions 19. As the first adhesive 60, an ultraviolet-curing type adhesive is preferably used from the standpoint of temporarily fixing the projection portions 19 and the opened side surface of the through hole 36b through instant curing.

The first adhesive 60 is supplied to a gap between the opened side surface of the through hole 36b and the projection portions 19 in order to bond the projection portions 19 and the opened side surface of the through hole 36b. Thus, the first adhesive 60 preferably has high viscosity (before curing) from the standpoint of preventing dripping of the first adhesive 60. Further, the first adhesive 60 preferably has high hardness after curing from the standpoint of strength and position accuracy of the temporary adhesion between the opened side surface of the through hole 36b and the projection portions 19.

The imaging module 20 and the light guide 40 are inserted into the through holes 36 and 38, respectively, to fix their relative positions and then the second adhesive 61 is filled into the through hole 36 to fix the positions of the imaging module 20 and the light guide 40. However, thermal expansion or thermal contraction at the time of curing the second adhesive 61 may cause a positional shift of the imaging module 20 in the through hole 36. It is conceivable that the imaging module 20 is temporarily fixed by the instantly cured first adhesive 60 and then adhesively fixed by the second adhesive 61 in order to prevent the positional shift of the imaging module 20. However, supplying the first adhesive 60 from the opening portion 36d without providing the projection portions 19 increases the use amount of the first adhesive 60 and causes the first adhesive 60 to spread inside the through holes 36 and enter the filling region of the second adhesive 61 used for permanently fixing. The second adhesive 61 has functions of not only fixing the position of the imaging module 20, but also reducing stress applied to the imaging module 20. Thus, it is not preferable to narrow the filling region of the second adhesive 61.

In the embodiment, providing the projection portions 19 to the outer periphery of the resin sealing portion 16 makes it possible to narrow a clearance between the imaging module 20 and the through hole 36 surface, reduce the use amount of the first adhesive 60, and prevent the first adhesive 60 from entering the filling region of the second adhesive 61. Further, the first adhesive 60 causes the imaging module 20 to be adhesively fixed to the frame body 30 in a state of covering the surface of the projection portions 19, making it possible to improve the connection strength in accordance with an increase in the connection area while reducing the use amount of the first adhesive 60.

The second adhesive 61 is filled in a gap between the through hole 36 and the imaging module 20 to bond the frame body 30 and the imaging module 20. After the projection portions 19 are adhesively fixed to the side surface of the through hole 36b by the first adhesive 60, the second adhesive 61 is supplied to a gap between the through hole 36 and the resin sealing portion 16 from the right and left opening portions 36d of the cured first adhesive 60. As the second adhesive 61, a thermosetting type adhesive is preferably used.

The second adhesive 61 preferably has low viscosity (before curing) from the standpoint of easily filling the second adhesive 61 into the opening portions 36d and the like. The viscosity of the second adhesive 61 before curing is preferably lower than that of the first adhesive 60.

The second adhesive 61 preferably has low hardness after curing from the standpoint of preventing a breakage of the imaging element 11 when stress is applied to the endoscope distal end structure 100, and the hardness of the second adhesive 61 after curing is preferably lower than that of the first adhesive 60 after curing.

In the embodiment, the first adhesive 60 causes the projection portions 19 to be adhesively fixed to the side surface of the through hole 36b, making it possible to prevent the positional shift of the imaging module 20 caused by thermal expansion or thermal contraction at the time of curing the second adhesive 61. Further, providing the projection portions 19 may reduce the use amount of the first adhesive 60 and prevent the first adhesive 60 from entering the filling region of the second adhesive 61.

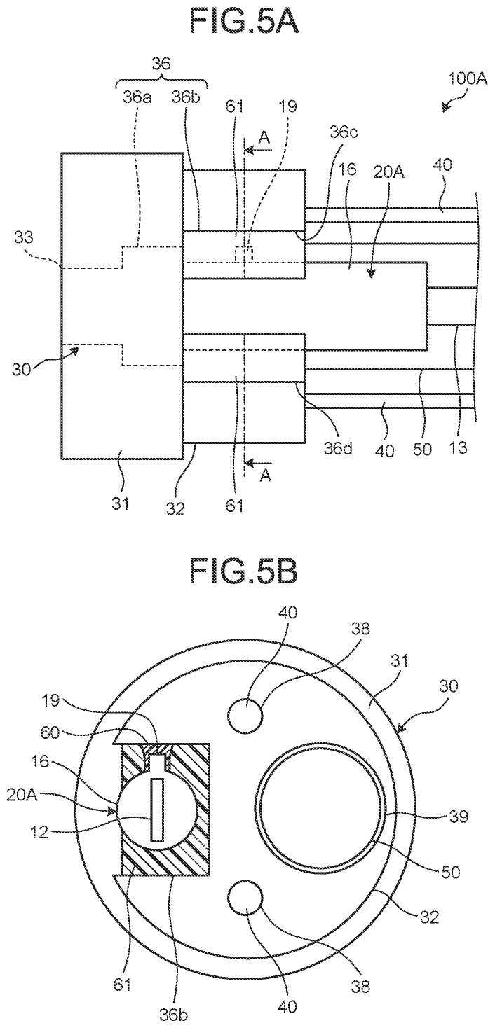

Two projection portions 19 are provided in the above embodiment. However, it is not necessary to provide two projection portions 19 and only one projection portion 19 may be formed. FIG. 5A illustrates a top view and FIG. 5B illustrates a cross sectional view taken along the line A-A in FIG. 5A of an endoscope distal end structure 100A according to a modification example 1 of the embodiment.

In an endoscope distal end structure 100A according to the modification example 1, only one projection portion 19 is formed on a resin sealing portion 16A of an imaging module 20A. When only one projection portion 19 is formed on one side, the position accuracy is slightly reduced as compared with a case where the projection portions 19 are Provided so as to be opposed to the side surface of the through hole 36b as described in the embodiment. However, it becomes possible to facilitate the filling work of the second adhesive 61 into the through hole 36b, prevent the positional shift of the imaging module 20A, and reduce the use amount of the first adhesive 60.

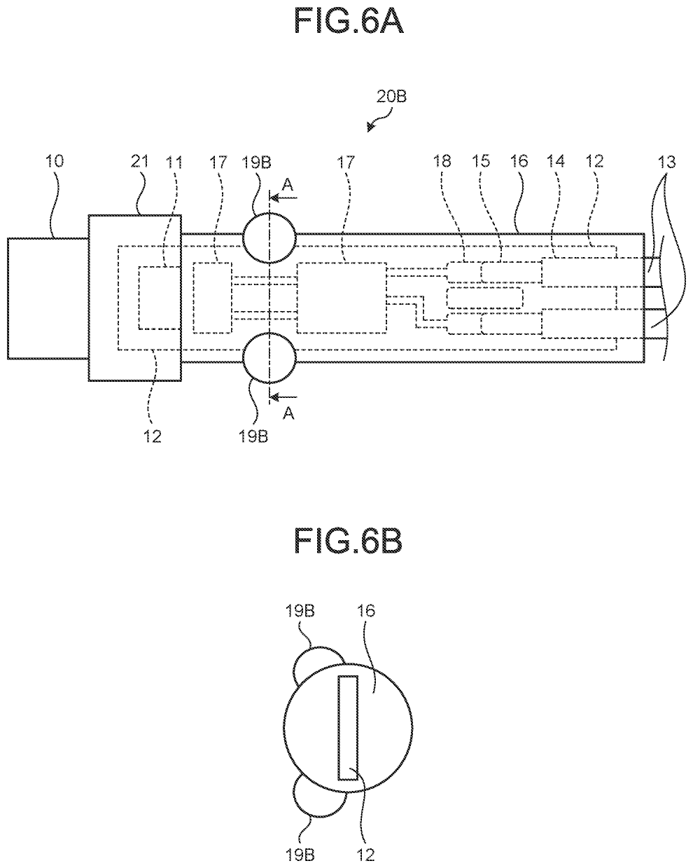

Further, in the above embodiment, the projection portions 19 are formed simultaneously with the resin sealing portion 16 using the same material as the resin sealing portion 16. However, the projection portions 19 may be formed separately. FIG. 6A illustrates a top view and. FIG. 6B illustrates a cross sectional view taken along the line A-A in FIG. 6A of an imaging module 20B according to a modification example 2 of the embodiment.

In an imaging module 20B according to the modification example 2, projection portions 19B are formed by applying an ultraviolet curing type adhesive to the side surface of the resin sealing portion 16 after forming the resin sealing portion 16 and then curing the adhesive by irradiation with ultraviolet rays. In the modification example 2, the resin sealing portion 16 and the projection portions 19B are formed without using a mold, making it simple to produce them.

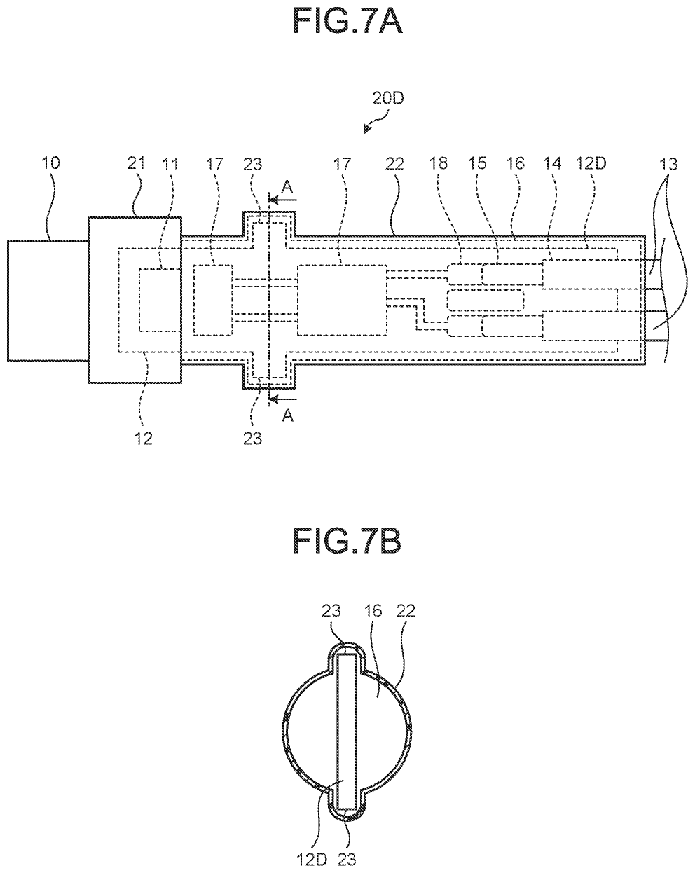

Further, in the above embodiment, the projection Portions 19 are formed on the resin sealing portion 16. However, the projection portions may be formed on the circuit substrate 12. FIG. 7A illustrates a top view and FIG. 7B illustrates a cross sectional view taken along the line A-A in FIG. 7A of an imaging module 20D according to a modification example 3 of the embodiment.

In an imaging module 20D according to a modification example 3, projection portions 23 are formed on the circuit substrate 12 using a substrate material of the circuit. substrate 12. In the imaging module 20D, the resin as a material of the resin sealing portion 16 is applied to a circuit substrate 12D including the projection portions 23 and, after curing, the resin sealing portion 16 is covered by a shrinkable tube 22. In the modification example 3, the resin sealing portion 16 and the projection portions 23 are formed without using a mold, making it simple to produce them.

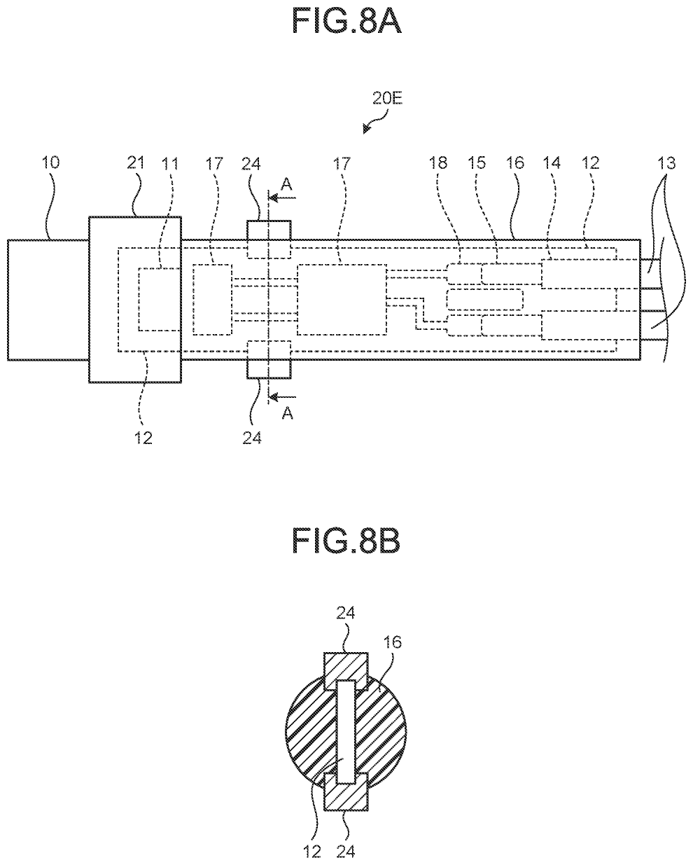

Further, the projection portions may be formed by metal pins. FIG. 8A illustrates a top view and FIG. 8B illustrates a cross sectional view taken along the line A-A in FIG. 8A of an imaging module 20E according to a modification example 4 of the embodiment.

In an imaging module 20E according to a modification example 4, projection portions 24 are formed by metal pins. The projection portions 24 are produced by inserting fitting metal pins to the circuit substrate 12 at the time of molding the resin sealing portion 16 using a mold and then fixing the metal pins to the circuit substrate 12 by the resin sealing portion 16. In the modification example 4, the projection portions 24 are formed by the metal pins, making it possible to improve rigidity and adhesive strength of a connection portion between the imaging module 20E and the frame body 30.

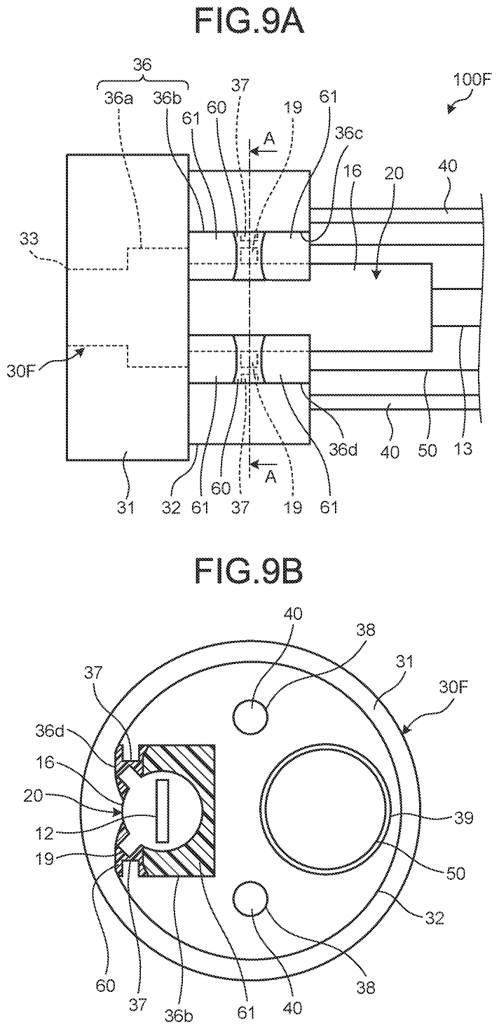

Further, in the present embodiment, the projection portions are provided on the outer periphery of the sealing resin of the imaging module. However, the projection portions may also be provided on the side surface of the through hole 36b. FIG. 9A illustrates a top view and FIG. 9B illustrates a cross sectional view taken along the line A-A in FIG. 9A of an endoscope distal end structure 100F according to a modification example 5 of the embodiment.

In an endoscope distal end structure 100F, projection portions 37 are formed on the side surface of the through hole 36b. The projection portions 37 are formed at positions opposite to the projection portions 19 when the imaging module 20 is inserted in the through hole 36 of a frame body 30F. Forming the projection portions 37 also on the frame body 30F side makes it easy, to align the positions of the frame body 30F and the imaging module 20 in addition to the advantageous effects of the embodiment. Further, the adhesive strength may improve due to an increase in the adhesive area.

According to the present disclosure, a positional shift of the imaging module in the frame body may be prevented, thus the imaging module may be installed in the frame body with a high position accuracy.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope distal end structure comprising:
an imaging module including
an optical unit,
an imaging element,
a circuit substrate,
an electronic component,
a cable,
a resin sealing portion configured to seal from an electronic-component mounting region of the circuit substrate to a mounting region of the cable, and
a projection portion protruding from an outer periphery of the resin sealing portion;
a frame body including a through hole, the through hole penetrating in an optical axis direction of the optical unit and having a partially opened side surface, and the through hole being configured to hold the imaging module in a state of being inserted is the through hole from an insertion port disposed at a proximal end portion of the through hole;
a first adhesive bonding the projection portion and the opened side surface of the through hole; and
a second adhesive filled in a gap between the through hole of the frame body and the imaging module inserted in the through hole and bonding the frame body and the imaging module.

2. The endoscope distal end structure according to claim 1, wherein the projection portion is formed on the outer periphery of the resin sealing portion visually recognizable from the partially opened side surface of the through hole.

3. The endoscope distal end structure according to claim 2, wherein two projection portions are formed at a same position in the optical axis direction of the side surface of the resin sealing portion so as to be opposed to the opened side surface of the through hole.

4. The endoscope distal end structure according to claim 1, wherein the second adhesive has lower hardness after curing than the first adhesive.

5. The endoscope distal end structure according to claim 1, wherein the first adhesive has higher viscosity before curing than the second adhesive.

6. The endoscope distal end structure according to claim 1, wherein the first adhesive is an ultraviolet-curing type adhesive and the second adhesive is a thermosetting type adhesive.

7. An endoscope comprising
an endoscope distal end structure disposed in as insertion portion, the endoscope distal end structure including:
an imaging module including
an optical unit,
an imaging element,
a circuit substrate,
an electronic component,
a cable,
a resin sealing portion configured to seal from an electronic-component mounting region of the circuit substrate to a mounting region of the cable, and
a projection portion protruding from as outer periphery of the resin sealing portion;
a frame body including a through hole, the through hole penetrating in an optical axis direction of the optical unit and having a partially opened side surface, and the through hole being configured to hold the imaging module in a state of being inserted in the through hole from an insertion port disposed at a proximal end portion of the through hole;
a first adhesive bonding the projection portion and the opened side surface of the through hole; and
a second adhesive filled in a gap between the through hole of the frame body and the imaging module inserted in the through hole and bonding the frame body and the imaging module.

8. The endoscope according to claim 7, wherein the projection portion is formed on the outer periphery of the resin sealing portion visually recognizable from the partially opened side surface of the through hole.

9. The endoscope according to claim 8, wherein two projection portions are formed at a same position in the optical axis direction of the side surface of the resin sealing portion so as to be opposed to the opened side surface, of the through hole.

10. The endoscope according to claim 7, wherein the second adhesive has lower hardness after curing than the first adhesive.

11. The endoscope according to claim 7, wherein the first adhesive has higher viscosity before curing than the second adhesive.

12. The endoscope according to claim 7, wherein the first adhesive is an ultraviolet-curing type adhesive and the second adhesive is a thermosetting type adhesive.
